(19) United States
(12) Patent Application Publication
(10) Pub. No.: US 2009/0030336 A1
(43) Pub. Date: Jan. 29, 2009

(54) APPARATUS AND DETECTING ANOMALY AND METHOD THEREOF

(75) Inventors: Eung Je Woo, Seongnam-si (KR); Dong In Oh, Yongin-si (KR); Jin Keun Seo, Seoul (KR); Oh In Kwon, Seoul (KR)

Correspondence Address: Workman Nydegger;1000 Eagle Gate Tower, 60 East South Temple, Salt Lake City, UT, 84111, US

(73) Assignee: University-Industry Cooperation Group of Kyunghee University, Yongin, Kyungki-do (KR)

(21) Appl. No.: 12/088,684

(22) PCT Filed: Jul. 28, 2006
(86) PCT No.: PCT/KR2006/002978

§ 371 Date: Mar. 29, 2008

(30) Foreign Application Priority Data
Apr. 14, 2006 (KR) 10-2006-0034080

(51) Int. Cl. A61B 5/053

(52) U.S. Cl. 600/547; 324/693

(57) ABSTRACT

An apparatus for detecting an anomaly and method thereof are disclosed, by which a size and location of an anomaly of a beast cancer and the like can be precisely detected as well as a presence or non-presence of the anomaly based on data measured on a surface of a body. The present invention includes supplying a first frequency voltage having a first frequency to a measurement target, detecting a first signal induced by the first frequency voltage from the measurement target, supplying a second frequency voltage having a second frequency to the measurement target, detecting a second signal induced by the second frequency voltage from the measurement target, correcting the first and second signals based on slopes of the detected first and second signals, and calculating a location and size of the anomaly within the measurement target based on the corrected first and second signals.

### TECHNICAL FIELD

The present invention relates to an apparatus for detecting an anomaly and method thereof. Although the present invention is suitable for a wide scope of applications, it is particularly suitable for detecting such an anomaly as a cancer, a tumor and the like using electrical characteristics of a body.

### BACKGROUND ART

Generally, women's breast cancers or large intestine cancers tend to keep increasing. Specifically, morbidity of breast cancer keeps increasing. In case of U.S.A., the breast cancer morbidity occupies 37.3% of morbidity of all kinds of cancers in 2001. In case of Japan, the breast cancer morbidity occupies 19.6% of morbidity of all kinds of cancers in 2001.

A most preferable treatment of cancer is to give corresponding medical treatment by detecting a tumor at an early stage before the tumor is transferred to another part of the body from an original site of disease. Yet, such an early diagnosis is possible in case of a presence of a secure and effective diagnostic technique. And, many efforts have been made to develop the early diagnosis of breast cancer using various techniques.

There are various early diagnostic techniques including X-ray mammography, ultrasonic photography, thermal infrared photography, CT, etc, for which various medical video equipments are used.

In the globally used X-ray mammography, it is difficult to diagnose a young woman's disease as a breast cancer due to X-ray characteristics since density of young woman's mammary glandular tissues is high. And, it is probable that the radioactivity used for the corresponding diagnosis may trigger a breast cancer.

In the ultrasonic photography, a video resolution is too low to be applied to an early diagnosis. And, the ultrasonic photography is still in dispute.

In case of the thermal infrared photography which uses a fact that a temperature of an inflamed or cancered cell is higher than that of a normal cell, a temperature of a skin surface is mainly shown on video. So, it is difficult to diagnose the breast cancer precisely.

In case of MRI or CT, a corresponding diagnostic cost is very high. And, it is difficult to photograph a woman's breast part only.

Recently, many efforts are made to detect breast cancer using a phenomenon that an electric conductivity of a normal tissue differs from that of a tumor tissue by three to ten times. So, a T-scan measurement technique is developed.

The T-scan measurement technique images a living tissue by measuring a current induced by a voltage applied to a human body. The T-scan measurement technique is identical to a measurement technique that uses a frontal plane impedance camera, which can be the first instance of early EIT (electrical impedance tomography) studied in the early 1980's. In particular, by applying a constant voltage to one portion of a human body via a surface electrode and maintaining a reference voltage of an electrode attached to another portion, a current bet measured to be imaged. And, a corresponding image is called a transfer admittance image. In T-scan, the transfer admittance image is outputted. And, a user decides a presence or non-presence of a patient's breast cancer through a subjective interpretation based on the outputted image.

However, the diagnostic equipment such as T-scan is not provided with a function of analyzing data of measurement of a breast surface. So, it is difficult to precisely detect an anomaly since a diagnostic result depends on a user's visual decision.

### DISCLOSURE OF THE INVENTION

Accordingly, the present invention is directed to an apparatus for detecting an anomaly and method thereof that substantially obviate one or more of the problems due to limitations and disadvantages of the related art.

An object of the present invention is to provide an apparatus for detecting an anomaly and method thereof, by which a size and location of an anomaly of a beast cancer and the like can be precisely detected as well as a presence or non-presence of the anomaly based on data measured on a surface of a body.

Additional features and advantages of the invention will be set forth in the description which follows, and in part will be apparent from the description, or may be learned by practice of the invention. The objectives and other advantages of the invention will be realized and attained by the structure particularly pointed out in the written description and claims thereof as well as the appended drawings.

To achieve these and other advantages and in accordance with the purpose of the present invention, as embodied and broadly described, a method of detecting an anomaly according to the present invention includes the steps of supplying a first frequency voltage having a first frequency to a measurement target, detecting a first signal induced by the first frequency voltage from the measurement target, supplying a second frequency voltage having a second frequency to the measurement target, detecting a second signal induced by the second frequency voltage from the measurement target, correcting the first and second signals based on slopes of the detected first and second signals, and calculating a location and size of the anomaly within the measurement target based on the corrected first and second signals.

Preferably, the step of calculating the location and size of the anomaly within the measurement target based on the corrected first and second signals includes the step of calculating the location of the anomaly within the measurement body based on a difference between the corrected first and second signals.

More preferably, the step of calculating the location of the anomaly within the measurement body based on a difference between the corrected first and second signals includes the steps of deciding an anomaly point on a detecting means corresponding to the location of the anomaly within the measurement target based on the difference between the corrected first and second signals and calculating a distance between the location of the anomaly within the measurement target and the anomaly point on the detecting means based on a distance between the anomaly point on the detecting means and a random point on the detecting means, a difference between the first and second signals detected at the anomaly point on the detecting means and a difference between the first and second signals detected at the random point on the detecting means.

Preferably, the step of calculating the location and size of the anomaly within the measurement target based on the corrected first and second signals includes the step of calculating the size of the anomaly within the measurement target based on a difference between the first and second signals detected on an anomaly point of a detecting means and a distance between the location of the anomaly within the measurement body and the anomaly point on the detecting means.

Preferably, the step of correcting the first and second signals based on the slopes of the detected first and second signals includes the steps of comparing each of the slopes of the first and second signals to a reference value and correcting the first and second signals according to a result of the comparing step.

Preferably, the step of supplying the second frequency voltage having the second frequency to the measurement target includes the step of if a supply of the first frequency voltage is terminated and if a time over one cycle of the first frequency voltage passes, supplying the second frequency voltage to the measurement target.

Preferably, the step of supplying the second frequency voltage having the second frequency to the measurement target includes the step of supplying the second frequency voltage having a frequency band higher than that of the first frequency voltage to the measurement target.

To further achieve these and other advantages and in accordance with the purpose of the present invention, an apparatus for detecting an anomaly includes a voltage generating unit supplying a first frequency voltage having a first frequency and a second frequency voltage having a second frequency to a measurement target, a detecting means for detecting a first signal induced by the first frequency voltage and a second signal induced by the second frequency voltage, a signal correcting unit correcting the first and second signals based on slopes of the detected first and second signals, and a control unit calculating a location and size of the anomaly within the measurement target based on the corrected first and second signals.

Preferably, the control unit calculates the location of the anomaly within the measurement body based on a difference between the corrected first and second signals.

More preferably, the control unit decides an anomaly point on the detecting means corresponding to the location of the anomaly within the measurement target based on the difference between the corrected first and second signals and calculates a distance between the location of the anomaly within the measurement target and the anomaly point on the detecting means based on a distance between the anomaly point on the detecting means and a random point on the detecting means, a difference between the first and second signals detected at the anomaly point on the detecting means and a difference between the first and second signals detected at the random point on the detecting means.

Preferably, the control unit calculates the size of the anomaly within the measurement target based on a difference between the first and second signals detected on an anomaly point of a detecting means and a distance between the location of the anomaly within the measurement body and the anomaly point on the detecting means.

Preferably, the signal correcting unit compares each of the slopes of the first and second signals to a reference value and then corrects the first and second signals according to a result of the comparing step.

Preferably, if a supply of the first frequency voltage is terminated and if a time over one cycle of the first frequency voltage passes, the voltage generating unit supplies the second frequency voltage to the measurement target.

Preferably, the voltage generating unit supplies the second frequency voltage having a frequency band higher than that of the first frequency voltage to the measurement target.

It is to be understood that both the foregoing general description and the following detailed description are exemplary and explanatory and are intended to provide further explanation of the invention as claimed.

### BRIEF DESCRIPTION OF DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 is a block diagram of an anomaly detecting apparatus according to an embodiment of the present invention;

FIG. 2 is a diagram for explaining a detecting method according to one embodiment of the present invention using an anomaly detecting apparatus of the present invention;

FIG. 3 is a block diagram of a constant voltage generating unit shown in FIG. 1;

FIG. 4 is a diagram of a scan probe according to one embodiment of the present invention;

FIG. 5 is an exemplary block diagram of a current measuring unit shown in FIG. 1;

FIG. 6 is another exemplary block diagram of a current measuring unit shown in FIG. 1; and

FIG. 7 is a flowchart of a method of detecting an anomaly according to the present invention.

### BEST MODE FOR CARRYING OUT THE INVENTION

Reference will now be made in detail to the preferred embodiments of the present invention, examples of which are illustrated in the accompanying drawings.

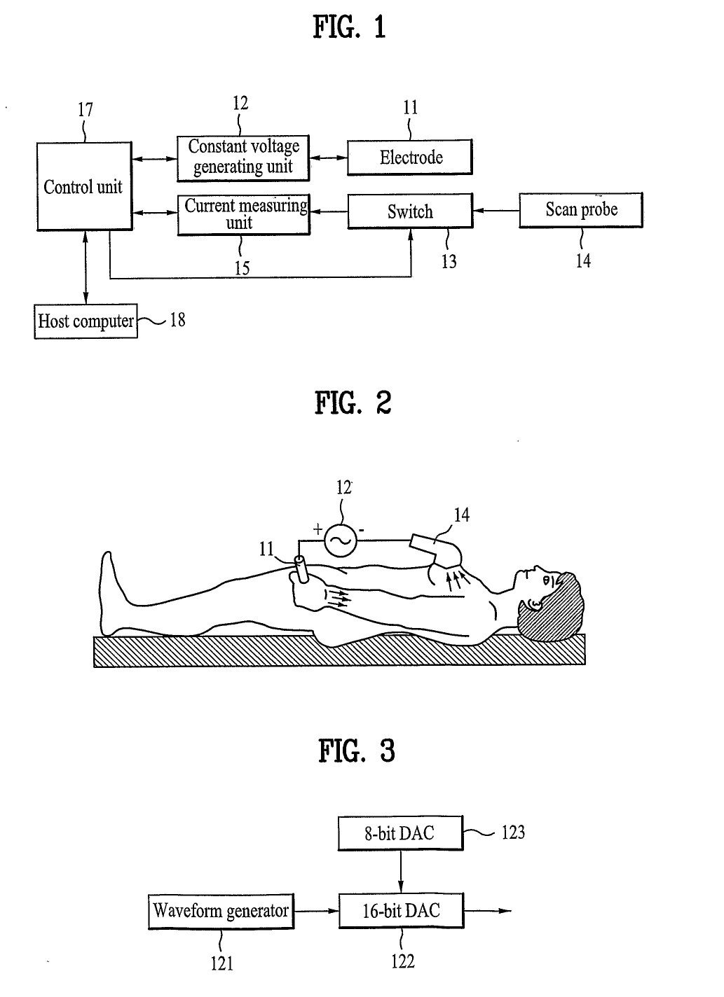

FIG. 1 is a block diagram of an anomaly detecting apparatus according to an embodiment of the present invention and FIG. 2 is an exemplary diagram for explaining a detecting method according to one embodiment of the present invention using an anomaly detecting apparatus of the present invention. In FIG. 1 and FIG. 2, elements required for explaining a technical idea of the present invention are shown only.

Referring to FIG. 2, a reference electrode 11 to apply a voltage is brought into contact with a portion of a body. And, the voltage is applied to the body via the reference electrode 11. For instance, the voltage is applied to an internal part of the body via such a body portion as a hand.

A constant voltage generating unit 12 supplies a voltage to the reference electrode 11. The constant voltage generating unit 12 generates at least one since wave constant voltage of a specific frequency and then provides the generated sine wave constant voltage to the reference electrode 11.

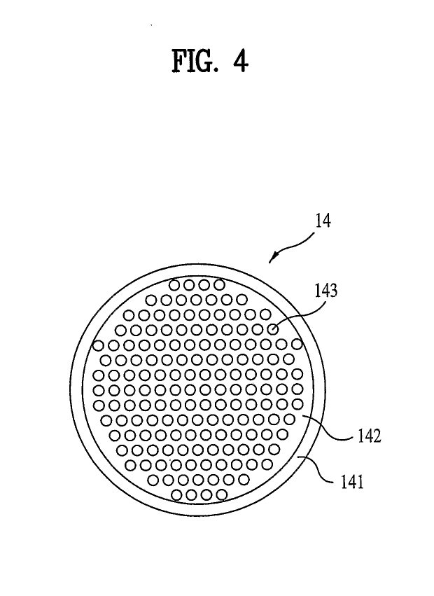

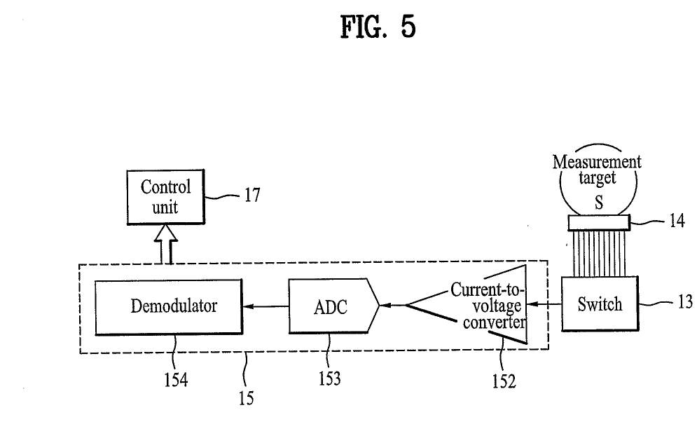

FIG. 3 is a block diagram of the constant voltage generating unit shown in FIG. 1.

Referring to FIG. 3, the constant voltage generating unit 12 includes a digital waveform generator 121 based on FPGA (field programmable gate array), a 16-bit digital-to-analog converter (DAC) 122 and an 8-bit digital-to-analog converter (DAC) 123.

The digital waveform generator 121 outputs a sine wave and is able to adjust a frequency of the sine wave in a range between 10 Hz and 500 KHz according to a command of a control unit 17. And, the 8-bit DAC 123 is able to adjust a size of the sine wave in a range between 0V and 2.5V. Hence, the constant voltage generating unit 12 is able to output the sine wave differing in size and frequency variously.

A scan probe 14 is brought into contact with a body portion to be checked and detects an electrical signal, i.e., a current flowing between the check portion and the reference electrode 11. For instance, in case of checking up a breast cancer, the scan probe 14 scans a current flowing through a chest.

And, the scan probe 14 includes a planar array of electrodes. Each of the planar array electrodes detects a current flowing through the check portion by maintaining a reference potential.

FIG. 4 is a diagram of a scan probe according to one embodiment of the present invention.

Referring to FIG. 4, a scan probe 14 includes a plurality of cylinder or disc type detect electrodes 143 periodically arranged with a predetermined gap in-between. For instance, the scan probe 14 includes 320 gold-plated detect electrodes 143. In this case, a diameter of each of the detect electrodes 143 is about 2 mm.

In order to arrange the detect electrodes 143 evenly spaced apart from one another, a shielding case 142 is needed to fix the detect electrodes 143 thereto. The shielding case 142 is formed of PCB. A plurality of holes are provided to the shielding case 142 to have the detect electrodes 143 inserted therein, respectively. And, the shielding case 142 is formed of an insulator to electrically isolate the detect electrodes 143 from one another.

A frame 141 formed of metal is provided to an outer circumference of the shielding case 142.

A current flowing from the reference electrode 11 to the scan probe 14 spreads through the entire body and then gathers into the scan probe 14. In this case, a current value inputted to the detect electrode 143 located in the vicinity of an outer side of the scan probe 14 is considerably greater than a current value inputted to another detect electrode 143 located at a center of the scan probe 14. So, an input value of the scan probe 14 is distorted. And, it is difficult to measure a microscopic current variation. To solve theses problems metal frame (guard electrode) is provided. The metal frame 141 enables the detect electrodes 143 and their peripheries to keep a reference potential (0V).

A current measuring unit 15 selectively receives currents detected by the detect electrodes 143 via a switch 13 and then measures a size of each of the detected currents.

FIG. 5 is an exemplary block diagram of the current measuring unit shown in FIG. 1.

Referring to FIG. 5, the current measuring unit 15 includes a current-to-voltage converter 152 converting the current received via the switch 13 to a voltage, an analog-to-digital converter (ADC) 153 converting an analog voltage signal outputted from the current-to-voltage converter 152 to a digital voltage signal and a digital phase detect demodulator measuring sizes of microscopic currents detected by the detect electrodes 143 based on the signal outputted from the analog-to-digital converter 153.

The AD153 converts the analog voltage signal to the digital voltage signal with 10 MHz sampling frequency.

In order to measure the sizes of the currents, the current measuring unit 15 can include a plurality of current measuring modules each of which includes at least one current-to-voltage converter 152, at least one ADC 153 and at least one demodulator 154.

For instance, if the current measuring unit 15 includes sixteen modules, each of the modules is able to be connected to a corresponding one of twenty detect electrodes 143 via the switch 13.

And, the current measuring unit 15 is able to further include a correction unit compensating a gain difference, a phase difference and the like off the current measuring modules.

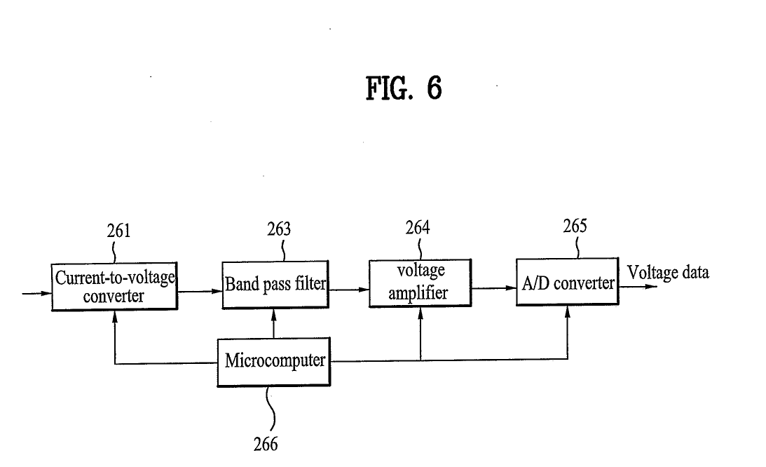

FIG. 6 is another exemplary block diagram of the current measuring unit shown in FIG. 1.

Referring to FIG. 6, the current measuring unit 15 can include a plurality of current measuring modules.

Each of a plurality of the current measuring modules includes a current-to-voltage converter 261, a band pass filter 263, a voltage amplifier 264, an A/D (analog/digital) converter 265 and a microcomputer 266.

The current-to-voltage converter 261 receives a current detected by each of the detect electrodes 143 according to a command of the microcomputer 266 and then converts the received current to a voltage.

A voltage signal outputted from the current-to-voltage converter 261 undergoes band path filtering by the band pass filter 263 and is then provided to the voltage amplifier 264 having a variable gain.

The voltage amplifier 264 includes a digital potentiometer having a maximum gain of 2,500.

A signal amplified by the voltage amplifier 264 is digitalized into a 10 MHz sampling frequency by the 12-bit A/D converter 265 according to a non-uniform sampling technique. By the non-uniform sampling technique, each amplified signal is sampled with a different timing for each of the amplified signals. For instance, assuming that one cycle of a signal is ‘1’, the signal is sampled for each of 0.1, 0.3, 0.5, 0.7 and 0.9 cycles during an odd cycle. And, the signal is sampled for each of 0.2, 0.4, 0.6, 0.8 and 1.0 cycles during an even cycle.

The microcomputer 266 is configured with FPGA and includes a half-duplex sync type serial port for infra-network, a spike noise removing digital filter and an auto-gain controller.

The microcomputer 266 computes a slope of voltage data inputted to the A/D converter 265 continuously. If the computed slope exceeds a threshold (slope of sine wave), the microcomputer 266 decides that noise is included in the voltage data. The microcomputer 266 then replaces the corresponding voltage data by a new value using the noise removing digital filter. For instance, the replacement value is an ideal value estimated based on a current value supplied to the reference electrode 11.

The microcomputer 266 detects a peak value of the voltage data during at least one cycle of a sine wave outputted from the A/D converter 265.

The microcomputer 266s a gain of the voltage amplifier 264 to have the detected peak value reach 90% of an output of the A/D converter 265 within a range between 1 and 2,500.

And, the microcomputer 266 controls a variable resistance included in the voltage amplifier 264 to adjust the gain (or, amplification rate) of the voltage amplifier 264. Besides, the control unit 17 is able to perform functions of the microcomputer 266 as well.

The control unit 17 outputs a clock signal to synchronize the constant voltage generating unit 12 and the current measuring unit 15 together. For instance, the control unit 17 transfers a clock signal of 40 MHz to the constant voltage generating unit 12 and the current measuring unit 15.

And, the control unit 17 is connected to a host computer 18 via a USB port and transfers current associated data measured by the current measuring unit 154 to the host computer 18. In this case, the host computer 18 includes a personal computer or a server connected to various anomaly detecting devices.

The host computer 18 detects a real-number component (in-phase) and an imaginary component (quadrature or out-of-phase) of each of the currents transferred by the control unit 17 and then computes transfer admittance data based on the detected components. Since current preferentially flows through such an anomaly as a cancer, a tumor and the like rather than a normal tissue, a presence or non-presence of anomaly can be decided using the transfer admittance data proportional to a size of current.

And, the host computer 18 computes a location and size of anomaly using the transfer admittance data and is then able to image a checked internal portion of body into a 3-dimensional image based on the computed values. This image offers information for conductivity and permittivity of a living tissue of the checked internal portion and enables a user to precisely know a location and size of anomaly as well as a presence or non-presence of such an anomaly as a cancer, a tumor and the like.

Alternatively, if the host computer 18 is not connected to an anomaly detecting device, the control unit 17 is able to perform a function of computing a location and size of anomaly based on the transfer admittance data.

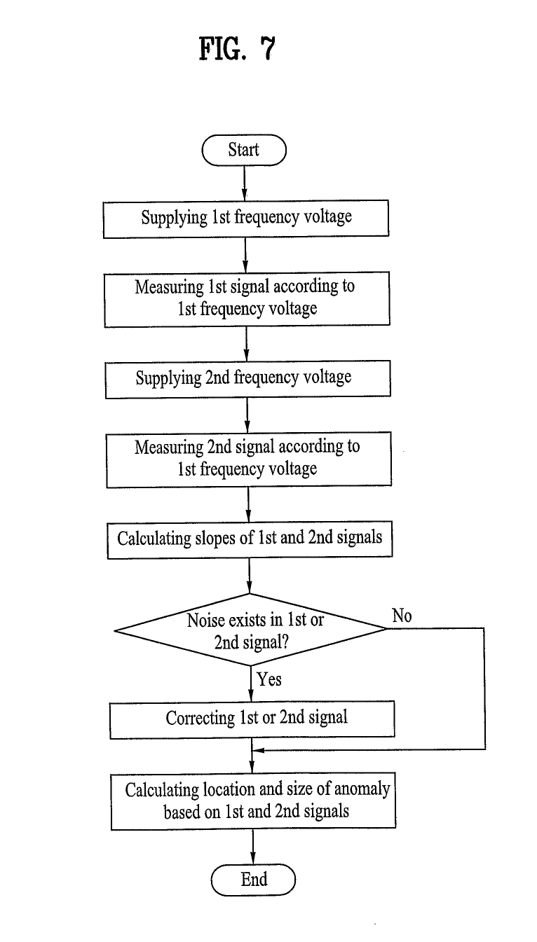

A method of detecting a location and size of anomaly based on the transfer admittance data is explained with reference to FIG. 7 as follows.

FIG. 7 is a flowchart of a method of detecting an anomaly according to the present invention.

Referring to FIG. 7, the constant voltage generating unit 12 generates a sine wave voltage of a first frequency according to a command of the control unit 17 and then transfers the generated voltage to the reference electrode 11 (S71). In this case, the constant voltage generating unit 12 outputs a sine wave voltage having a frequency corresponding to a range between 10 Hz and 500 KHz, e.g., a 50 KHz sine wave voltage.

If the sine wave voltage of the first frequency is introduced into a measurement target via the reference electrode 11, each of the detect electrodes 143 of the scan probe 14 detects a current at a portion to be examined (S72).

Subsequently, the current detected by each of the detect electrodes 143 is transferred to the current measuring unit 15. The current measuring unit 15 then measures levels of the currents detected by the detect electrodes 143, respectively. The current measuring unit 15 delivers all real-number and imaginary number components of the measured current values to the control unit 17.

If so, the control unit 17 transfers the measured current values to the host computer 18.

After a predetermined time has passed, the constant voltage generating unit 12 outputs a sine wave voltage of a second frequency (e.g., 500 MHz) different from the first frequency (50 MHz) of the former sine wave voltage. After the output of the sine wave voltage of the first frequency has been terminated, the constant voltage generating unit 12 outputs the sine wave voltage of the second frequency if a time corresponding to a prescribed cycle (e.g., two cycles (180°)) of the sine wave voltage of the first frequency is passed or if a sum of the currents detected by the detect electrodes 143 becomes zero (S73).

In the same manner, the sine wave voltage of the second frequency is inputted to the measurement target and a current according to the sine wave voltage of the second frequency is then detected (S74).

The current measuring unit 15 calculates a slope of a first current signal attributed to the first frequency voltage and a slope of a second current signal attributed to the second frequency voltage (S75).

Based on the slopes of the calculated first and second current signals, the current measuring unit 15 decides whether noise exists in each of the first and second current signals (S76). For instance, each of the slopes of the first and second current signals is compared to a threshold and a presence or non-presence of noise is then decided according to a result of the comparison. If the noise exists in the first or second current signal, the current measuring unit 15 corrects the first or second current signal to remove the corresponding noise component from the corresponding signal (S77).

Optionally, the current measuring unit 15 may determine whether noise exists in the first current signal before the constant voltage generating unit 12 outputs the second frequency voltage.

Subsequently, a location and size of an anomaly is computed based on the first and second current signals (S78). Transfer admittance data is computed based on current values generated from the sine wave voltages differing from each other in frequency. Algorithm for computing the location and size of the anomaly based on the computed transfer admittance data is explained as follows.

First of all, in case of using two sine wave voltages differing from each other in frequency, let's assume that transfer admittance distributions corresponding to two frequencies ω and ω′ are g(x,y) and g′(x,y), respectively and that complex conductivities of normal tissue and tumor tissue corresponding to the frequency ω′ are τ′₁=σ₁+jω′ε₁ and τ′₂=σ₂+jω′ε₂, respectively.

A relation between a location and size of anomaly and the transfer admittance distributions corresponding to the two frequencies is defined by Formula 1.

$${g}^{′} ( x , y ) - g ( x , y ) = A \frac{18 α ( {τ}_{2} - {τ}_{2}^{′} )}{( 2 {τ}_{1} + {τ}_{2} ) ( 2 {τ}_{1}^{′} + {τ}_{2}^{′} )} \frac{2 {d}^{2} - {( x - {ξ}_{1} )}^{2} - {( y - {ξ}_{2} )}^{2}}{4 {π [ \begin{matrix}{( x - {ξ}_{1} )}^{2} - \\ {( y - {ξ}_{2} )}^{2} + {d}^{2}\end{matrix} ]}^{5 / 2}} \qquad [ Formula 1 ]$$

In this case, symbols used in Formula 1 are as follows:

‘A’ is a volume of anomaly;

‘(x,y)’ is a random point in a plane Γ of the scan probe 14;

‘α’ is a mean value of transfer admittance distribution g(x,y) in the plane of the scan probe 14;

‘(ξ₁, ξ₂)’ is a point on the plane of the scan probe 14 corresponding to an anomaly within a body; and

‘d’ is a vertical distance from a point (ξ₁, ξ₂) in the plane of the scan probe 14 to a weight center of anomaly.

In τ₁=σ₁+jωε₁, σ₁ is a mean conductivity of a normal tissue, ε₁ is a mean permittivity of a normal tissue, and ω is a frequency of a sine wave voltage.

In τ₂=σ₂+jωε₂, σ₂ is a mean conductivity of a tumor tissue, ε₂ is a mean permittivity of a tumor tissue, and ω is a frequency of a sine wave voltage.

$$( {ξ}_{1} , {ξ}_{2} ) = {arg max}_{( x , y ) H {Γ}_{L}} \vert {g}^{′} ( x , y ) - g ( x , y ) \vert \qquad [ Formula 2 ]$$

By Formula 2, a point (ξ₁, ξ₂) in the plane of the scan probe 14 corresponding to a location of an anomaly can be found. The point (ξ₁, ξ₂) in the plane of the scan probe 14 is the point at which the absolute value |g′(x,y)−g(x,y)| has a maximum value in the plane of the scan probe 14. In order to find the point (ξ₁, ξ₂), the control unit 17 compares the admittance distribution g′(x,y) corresponding to the frequency ω′ and the admittance distribution g(x,y) corresponding to the frequency ω to each other. The control unit 17 then decides a point, which has a maximum value among the differences between the two admittance distributions, in the plane of the scan probe 14 as the point (ξ₁, ξ₂).

$$\vert \frac{{g}^{′} ( {ξ}_{1} - {ξ}_{2} ) - g ( {ξ}_{1} - {ξ}_{2} )}{{g}^{′} ( x , y ) - g ( x , y )} \vert = \frac{\vert 2 - \frac{{l}^{2}}{{d}^{2}} \vert}{2 {( \frac{{l}^{2}}{{d}^{2}} + 1 )}^{5 / 2}} \qquad [ Formula 3 ]$$

By Formula 3, a depth d of anomaly can be found. In Formula 3, (x,y) is a random point in the plane of the scan probe 14 in the vicinity of the point (ξ₁, ξ₂) a distance l is a distance between the points (x,y) and (ξ₁, ξ₂), i.e., l=\sqrt{(x−ξ₁)²+(y−ξ₂)²}. The control unit 17 calculates the distance l between the points (x,y) and (ξ₁, ξ₂) . The control unit 17 then calculates a distance d to an actual anomaly from the point (ξ₁, ξ₂) in the plane of the scan probe 14 based on the two admittance values g′(ξ₁−ξ₂) and g(ξ₁−ξ₂) measured at the point (ξ₁, ξ₂), the two admittance values g′(x,y) and g(x,y) measured at the random point (x,y) and the calculated distance l. Like Formula 3, it can be seen that the distance d varies according to a ratio of the difference g′(x,y)−g(x,y) between the two admittance values to the difference between the two admittance values g′(ξ₁−ξ₂)−g(ξ₁−ξ₂) and that the distance d varies according to the distance l. Once the point (ξ₁, ξ₂) in the plane of the scan probe 14 corresponding to the location of the anomaly and the distance d to the anomaly from the point (ξ₁, ξ₂) are known, it is able to know a precise location of the anomaly existing within a body.

$$A = \frac{π \vert 2 {τ}_{1} + {τ}_{2} \vert \vert 2 {τ}_{1}^{′} + {τ}_{2}^{′} \vert \vert {g}^{′} ( {ξ}_{1} - {ξ}_{2} ) - g ( {ξ}_{1} - {ξ}_{2} ) \vert {d}^{3}}{9 \vert α \vert \vert {τ}_{2} - {τ}_{2}^{′} \vert} \qquad [ Formula 4 ]$$

A size (volume) of anomaly can be found by Formula 4. Once the distance d to the anomaly from the point (ξ₁, ξ₂) in the plane of the scan probe 14 is calculated, the control unit 17 is able to find the volume A of the anomaly. As expressed in Formula 4, the volume A of the anomaly depends on the distance d, the difference g′(ξ₁−ξ₂)−g(ξ₁−ξ₂) between the two admittance values and the mean value α of the transfer admittance distribution in the plane of the scan probe 14 and also depends on the conductivity and permittivity associated data τ₁, τ₂, τ′₁ and τ′₂ corresponding to the two sine wave voltages.

### INDUSTRIAL APPLICABILITY

Accordingly, the present invention provides the following effects or advantages.

First of all, the present invention is able to decide whether an anomaly exists within a body based on admittance data and detect a location and size of the anomaly.

Secondly, the present invention is able to manufacture an instrument less harmful and less expensive than a conventional breast cancer diagnostic instrument detecting a breast cancer.

Thirdly, since constant voltages differing from each other in frequency are sequentially applied to a body with a prescribed duration, it is able to precisely detect a location and size of an anomaly without noise generated from interference between two constant voltages.

Therefore, the present invention is usefully applicable to the diagnosis of breast cancer.

While the present invention has been described and illustrated herein with reference to the preferred embodiments thereof, it will be apparent to those skilled in the art that various modifications and variations can be made therein without departing from the spirit and scope of the invention. Thus, it is intended that the present invention covers the modifications and variations of this invention that come within the scope of the appended claims and their equivalents.

1. A method of detecting an anomaly, comprising the steps of:
supplying a first frequency voltage having a first frequency to a measurement target;
detecting a first signal induced by the first frequency voltage from the measurement target;
supplying a second frequency voltage having a second frequency to the measurement target;
detecting a second signal induced by the second frequency voltage from the measurement target;
correcting the first and second signals based on slopes of the detected first and second signals; and
calculating a location and size of the anomaly within the measurement target based on the corrected first and second signals.

2. The method of claim 1, the step of calculating the location and size of the anomaly within the measurement target based on the corrected first and second signals, comprising the step of calculating the location of the anomaly within the measurement body based on a difference between the corrected first and second signals.

3. The method of claim 2, the step of calculating the location of the anomaly within the measurement body based on a difference between the corrected first and second signals, comprising the steps of:
deciding an anomaly point on a detecting means corresponding to the location of the anomaly within the measurement target based on the difference between the corrected first and second signals; and
calculating a distance between the location of the anomaly within the measurement target and the anomaly point on the detecting means based on a distance between the anomaly point on the detecting means and a random point on the detecting means, a difference between the first and second signals detected at the anomaly point on the detecting means and a difference between the first and second signals detected at the random point on the detecting means.

4. The method of claim 1, the step of calculating the location and size of the anomaly within the measurement target based on the corrected first and second signals, comprising the step of calculating the size of the anomaly within the measurement target based on a difference between the first and second signals detected on an anomaly point of a detecting means and a distance between the location of the anomaly within the measurement body and the anomaly point on the detecting means.

5. The method of claim 1, the step of correcting the first and second signals based on the slopes of the detected first and second signals, comprising the steps of:
comparing each of the slopes of the first and second signals to a reference value; and
correcting the first and second signals according to a result of the comparing step.

6. The method of claim 1, the step of supplying the second frequency voltage having the second frequency to the measurement target, comprising the step of if a supply of the first frequency voltage is terminated and if a time over one cycle of the first frequency voltage passes, supplying the second frequency voltage to the measurement target.

7. The method of claim 1, the step of supplying the second frequency voltage having the second frequency to the measurement target, comprising the step of supplying the second frequency voltage having a frequency band higher than that of the first frequency voltage to the measurement target.

8. An apparatus for detecting an anomaly, comprising:
a voltage generating unit supplying a first frequency voltage having a first frequency and a second frequency voltage having a second frequency to a measurement target;
a detecting means for detecting a first signal induced by the first frequency voltage and a second signal induced by the second frequency voltage;
a signal correcting unit correcting the first and second signals based on slopes of the detected first and second signals; and
a control unit calculating a location and size of the anomaly within the measurement target based on the corrected first and second signals.

9. The apparatus of claim 8, wherein the control unit calculates the location of the anomaly within the measurement body based on a difference between the corrected first and second signals.

10. The apparatus of claim 9, wherein the control unit decides an anomaly point on the detecting means corresponding to the location of the anomaly within the measurement target based on the difference between the corrected first and second signals and calculates a distance between the location of the anomaly within the measurement target and the anomaly point on the detecting means based on a distance between the anomaly point on the detecting means and a random point on the detecting means, a difference between the first and second signals detected at the anomaly point on the detecting means and a difference between the first and second signals detected at the random point on the detecting means.

11. The apparatus of claim 8, wherein the control unit calculates the size of the anomaly within the measurement target based on a difference between the first and second signals detected on an anomaly point of a detecting means and a distance between the location of the anomaly within the measurement body and the anomaly point on the detecting means.

12. The apparatus of claim 8, wherein the signal correcting unit compares each of the slopes of the first and second signals to a reference value and then corrects the first and second signals according to a result of the comparing step.

13. The apparatus of claim 8, wherein if a supply of the first frequency voltage is terminated and if a time over one cycle of the first frequency voltage passes, the voltage generating unit supplies the second frequency voltage to the measurement target.

14. The apparatus of claim 8, wherein the voltage generating unit supplies the second frequency voltage having a frequency band higher than that of the first frequency voltage to the measurement target.
